(19) United States
(12) Patent Application Publication
(10) Pub. No.: US 2018/0015111 A1
(43) Pub. Date: Jan. 18, 2018

(54) METHODS FOR ON DEMAND PRE-EXPOSURE PROPHYLACTIC TREATMENT OF HIV INFECTION

(71) Applicants: INSERM (INSTITUT NATIONAL DE LA SANTE ET DE LA RECHERCHE MEDICALE), Paris (FR); COMMISSARIAT À L'ÉNERGIE ATOMIQUE, Paris (FR)

(72) Inventors: Jean-Michel MOLINA, Paris Cedex 10 (FR); Jean-Francois DELFRAISSY, Fontenay aux Roses (FR); Bruno SPIRE, Marseille (FR); Jean-Pierre ABOULKER, Villejuif (FR)

(21) Appl. No.: 15/551,773

(22) PCT Filed: Feb. 18, 2016
(86) PCT No.: PCT/EP2016/053456

§ 371 Date: Aug. 17, 2017

(30) Foreign Application Priority Data
Feb. 18, 2015 (EP) 15305243.6

(51) Int. Cl. A61K 31/675; A61K 31/513

(52) U.S. Cl. CPC A61K 31/675; A61K 31/513; A61K 2300/00

(57) ABSTRACT

The present invention relates to methods for on demand pre-exposure prophylactic treatment of HIV infection. In particular, the present invention relates to a method for on demand pre-exposure prophylactic treatment of a subject at substantial risk for HIV infection comprising administering orally the subject with a combination of a therapeutically effective amount of emtricitabine and tenofovir prior and after to exposure to a potential source of HIV.

### FIELD OF THE INVENTION

The present invention relates to methods for on demand pre-exposure prophylactic treatment of HIV infection.

### BACKGROUND OF THE INVENTION

In its publication on monitoring HIV infections in France in 2008, the French Institute for Public Health Surveillance (InVS) estimated approximately 6940 (CI 95%: 6200-7690) new cases of contamination with HIV in France (Le Vu S, Le Strat Y, Barin F, et al. Population based HIV incidence in France 2003-08: a modelling analysis. Lancet Infectious Diseases 2010; 10:682-7.). This number of new contaminations is to be balanced against the number of AIDS declarations during the same period, which was not more than approximately 1550 cases per year in 2008. Therefore, this is an evidence of the insufficiency of current preventative measures for HIV infection in France and of the need to reinforce this prevention. Moreover, whereas this number of new declarations of infections with HIV has decreased overall in France since 2003 in all at-risk groups, it remains stable, or has even increased in the group of male homosexuals, which alone represents 48% of all new contaminations in 2008, or approximately 3320 cases (CI 95%: 2830-3810). This high number of new contaminations with HIV in male homosexuals is related to a parallel increase in sexually transmitted diseases (STD: infections with chlamydia, syphilis), which attests to the fact that, in these subjects, there is a persistence of at-risk sexual behaviour as regards HIV and suggests that, indeed, the number of new HIV contaminations is on the rise. Therefore, this incidence of new HIV contaminations in homosexuals in France is worrisome, and shows that preventative actions and specific and new screening methods need to be undertaken in an urgent manner. In fact, this incidence of new contaminations is close to and perhaps higher than that in countries strongly endemic for HIV in sub-Saharan Africa.

New approaches to the prevention of HIV infection are, therefore, necessary in order to consider the limits of current strategies. Among the preventative measures that could be proposed to these people, pre-exposure antiretroviral treatment (PrEP) may find its place. In fact, there are several studies underway throughout the world at the moment to evaluate the efficacy of this antiretroviral-based PrEP strategy in preventing infection with HIV in human subjects. In all cases the strategies are evaluated based on continuous oral antiretroviral treatment in combination with tenofovir or tenofovir and FTC versus placebo. In fact, regardless of the final results of these studies, it is not very likely that a PrEP continuous treatment strategy will be used in practice, considering the constraints of permanent antiretroviral treatment, its potential adverse effects and its cost. In reality, it seems more pertinent to evaluate a PrEP “on demand” strategy, in sync with sexual exposure, since this is the manner in which PrEP will probably be taken by subjects if it should become available. Even though it is possible for an “on demand” prophylactic treatment to be intrinsically less effective than continuous prophylactic treatment, adherence to and tolerance of this type of strategy may be better with a more attractive benefit-risk and cost-efficacy ratio. In addition, the data obtained in animals is in favour of the efficacy of this type of intermittent PrEP strategy (e.g Garcia-Lerma et al. Intermittent prophylaxis with oral Truvada protects macaques from rectal SHIV infection. Science Translational Medicine, 2010; 2; 14ra4).

### SUMMARY OF THE INVENTION

The present invention relates to methods for on demand pre-exposure prophylactic treatment of HIV infection. In particular, the present invention is defined by the claims.

### DETAILED DESCRIPTION OF THE INVENTION

Daily PrEP with oral TDF-FTC can reduce the risk of HIV infection in high risk individuals but long term adherence to a daily regimen remains challenging and explains the discordant results reported across trials. Now the inventors demonstrate that on demand PrEP with oral TDF-FTC is highly effective to reduce the incidence of HIV-infection in particular in high risk MSM and has a good safety profile.

Accordingly, the present invention relates to a method for on demand pre-exposure prophylactic treatment of a subject at substantial risk for HIV infection comprising administering orally the subject with a combination of a therapeutically effective amount of emtricitabine and tenofovir prior and after exposure to a potential source of HIV.

The terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease (i.e. a source of HIV). In particular, the “prophylactic treatment” as used in the context of the invention is defined by the subject being serologically negative after being exposed to the source of HIV.

In some embodiments, potential sources of HIV include sexual intercourse, medical worker skin puncture inoculation, hypodermic needle sharing, blood transfusions, birth canal exposure, breastfeeding, and transplacental contact between individuals. When the subject is at risk during sexual transmission, this includes anyone who is in an ongoing relationship with an HIV-positive partner. In a particular embodiment, the potential source of HIV is sexual intercourse.

It also includes anyone who 1) is not in a mutually monogamous relationship with a partner who recently tested HIV-negative, and 2) is a gay or bisexual man who has had anal sex without a condom or been diagnosed with an STD in the past 6 months; or heterosexual man or woman who does not regularly use condoms during sex with partners of unknown HIV status who are at substantial risk of HIV infection (e.g., people who inject drugs or have bisexual male partners). In some embodiments, the present invention is particular suitable for the on demand pre-exposure prophylactic treatment of high-risk adults, and in particular men who have sex with men.

As used herein, the term “tenofovir” has its general meaning in the art and refers to ({[(2R)-1-(6-amino-9H-purin-9-yl)propan-2-yl]oxy}methyl)phosphonic acid.

As used herein, the term “emtricitabine” has its general meaning in the art and refers to 4-amino-5-fluoro-1-[(2R,5S)-2-(hydroxymethyl)-1,3-oxathiolan-5-yl]-1,2-dihydropyrimidin-2-one.

The combination treatment according to the invention is administrated in the form of a pharmaceutical composition suitable for oral route. Suitable oral-route forms such as tablets, gel capsules, powders, granules and oral suspensions or solutions. In some embodiments, tenofovir and emtricitabine is combined in one pill. Typically, the pill is commercially available from Gilead in the name of Truvada®. In some embodiments, the pill consists of 300 milligrams of tenofovir disoproxil fumarate (of which 245 mg tenofovir) and 200 milligrams of emtricitabine.

The combination of tenofovir and emtricitabine is provided through a dosing regimen. A dosing regimen according to the present invention typically includes one dose administered prior to exposure and two doses after exposure. In some embodiments, the subject is administered with a first dose of the combination 24 h before the exposure and then receives a second dose 24 h after and a third dose after 48 h after first intake.

One important feature of the present invention is that the subject is not administered chronically with the combination (e.g. every day) and receive the combination treatment “on demand” just before to be exposed to the source of HIV, notably in view of a sexual intercourse. As used herein, the term “on demand” refers the ability to allow a subject to initiate the combination treatment of the present invention at any desired time before and after to be exposed to source of HIV.

It is appreciated that establishing a therapeutic concentration at the time of exposure includes factors for the therapeutic agent such as the route of administration, pharmacokinetics, absorption rate based on administration route, effects of food on oral absorption, in vivo distribution, metabolic pathways, elimination route, race, gender, and age of the subject, single dose incident side effects, long term administration side effects, and synergistic effects with co-administered active agents. Typically, the subject takes 2 pills of tenofovir-emtricitabine 24 h prior to the exposure and then another pill 24 h later and a fourth pill 48 h after the first drug intake. Particularly, the subject takes 2 pills of tenofovir-emtricitabine 24 h prior to a sexual intercourse, and then a third pill 24 h after the sexual intercourse and a fourth pill 48 h after the first drug intake.

The invention will be further illustrated by the following figures and examples. However, these examples and figures should not be interpreted in any way as limiting the scope of the present invention.

### FIGURES

FIG. 1: Enrollment and Follow-up of the Study Participants. The most common reasons for ineligibility were ongoing HIV-1 infection and laboratory abnormalities. Only 2 participants met one of the noninclusion criteria of a creatinine clearance of less than 60 ml per minute, glycosuria, or proteinuria, all of which were designed to minimize potential renal toxic effects from exposure to tenofovir disoproxil fumarate (TDF). A total of 14 participants (3 with HIV-1 infection, 6 who withdrew consent, and 5 who were lost to follow-up) underwent randomization but were not enrolled, and their data were not included in the primary modified intention-to-treat analysis. Attendance at clinic visits is shown on a quarterly basis for all participants who remained in the study. Study visits were scheduled 4 and 8 weeks after enrollment and every 8 weeks thereafter. FTC denotes emtricitabine.

FIG. 2: Kaplan-Meier Estimates of the Probability of HIV-1 Infection. The cumulative probability of HIV-1 acquisition is shown for the two study groups in the modified intention-to-treat analysis. The inset shows the same data on an enlarged y axis.

### EXAMPLES

### Example 1

Methods: High risk adult MSM who reported condomless anal sex and had a creatinine clearance >60 mL/mn were enrolled in this prospective randomized double-blinded placebo-controlled study. Participants (pts) were asked to take two pills of TDF-FTC (300 mg/200 mg per pill) or placebo 2 to 24 h before each sexual intercourse, then another pill 24 h later and a fourth pill 48 h after the first drug intake. All subjects received risk-reduction counseling, condoms, HBV and HAV vaccines when needed, were informed about post-exposure prophylaxis and were regularly tested for HIV and other sexually transmitted infections (STIs). The primary study objective was to demonstrate a reduction in HIV incidence with on demand PrEP. In November 2014, following the DSMB recommendation, the placebo arm was discontinued and on demand PrEP was offered to all participants.

Results: From February 2012 to November 2014, 414 pts were randomized and 400 without HIV-infection were enrolled. After a median follow-up of 8.8 months (IQR: 4.3 to 20.5), the incidence of HIV-infection was 6.75 per 100 pt-years in the placebo arm and 0.94 per 100 pt-years in the TDF-FTC arm indicating a relative reduction of 86% in the incidence of HIV with on demand PrEP (95% CI: 39.4-98.5%, P=0.002). Sixteen pts acquired HIV-infection after enrollment, 14 in the placebo arm and 2 in the TDF-FTC arm. Pts used a median of 14 pills/month (IQR: 8-20). Overall, 34% of pts acquired a new STIs. Safety was good with only one pt discontinuing TDF-FTC because of suspected drug-drug interaction. The rate of serious adverse events was low (9%) and similar across the study arms. Drug-related gastrointestinal adverse events (nausea, diarrhea, abdominal pain) were reported more frequently with TDF-FTC than with placebo (13% vs 6%, p=0.02). Only 2 pts (1%) in the TDF-FTC arm had transient decreases in creatinine clearance <60 mL/mn.

Conclusion: On demand PrEP with oral TDF-FTC is highly effective to reduce the incidence of HIV-infection in high risk MSM and has a good safety profile.

### Example 2

Methods:

Protocol and Study Population

The protocol was approved by public health authorities and by ethics committees in France (Comité de Protection des Personnes Ile de France IV) and Canada (Comité d'Ethique de la Recherche de Montreal). All participants provided written informed consent. Full details with respect to the study design can be found in the study protocol, available with the full text of this article at NEJM.org.

Inclusion criteria were HIV-negative status, an age of at least 18 years, and male or transgender female sex among participants who have sex with men and who are at high risk for HIV infection (defined as a history of unprotected anal sex with at least two partners during the past 6 months). Exclusion criteria included positive results on testing for hepatitis B surface antigen, chronic infection with hepatitis C virus, a creatinine clearance of less than 60 ml per minute (as assessed by means of the Cockroft-Gault equation), an alanine aminotransferase level of more than 2.5 times the upper limit of the normal range, and glycosuria or proteinuria of more than 1+ on urine dipstick testing.

Randomization and Study Procedures

Randomization was performed by means of a fixed-size block of 4 and stratified according to country. At enrollment, eligible HIV-negative participants were assigned in a 1:1 ratio to receive either TDF-FTC or placebo. The use of a placebo was deemed to be justified because of the inconsistent efficacy of preexposure prophylaxis in previous trials and the moderate efficacy of preexposure prophylaxis in the iPrex trial among men who have sex with men.

TDF-FTC was given as a fixed-dose combination of 300 mg of TDF and 200 mg of FTC per pill. Participants were instructed to take a loading dose of two pills of TDF-FTC or placebo with food 2 to 24 hours before sex, followed by a third pill 24 hours after the first drug intake and a fourth pill 24 hours later. In case of multiple consecutive episodes of sexual intercourse, participants were instructed to take one pill per day until the last sexual intercourse and then to take the two postexposure pills. When resuming preexposure prophylaxis, participants were instructed to take a loading dose of two pills unless the last drug intake was less than 1 week earlier, in which case they were instructed to take only one pill.

Study visits were scheduled 4 and 8 weeks after enrollment and every 8 weeks thereafter. Each visit included drug dispensation with enough pills to cover the daily use of TDF-FTC or placebo between visits, pill count and adherence counseling, serum testing for HIV-1 and HIV-2, and biochemical analyses. Before each visit, participants were asked to complete at home a computer-assisted structured interview to collect information about sociodemographic characteristics, use of alcohol and recreational drugs, sexual behavior, and adherence to pre-exposure prophylaxis during their most recent sexual intercourse.

Standard Prevention Interventions

At every scheduled visit, participants were offered a comprehensive package of prevention services, including patient-centered, interactive counseling according to the RESPECT risk-reduction model performed by a peer community member, free condoms and gel, and diagnosis and treatment of sexually transmitted infections. Peer counselors were also available between visits to address participants' needs and reinforce adherence to study medications. Vaccination against hepatitis A and B was offered to all participants who were at risk for these infections. At enrollment and every 6 months thereafter, participants were screened for syphilis (on serologic analysis) and for chlamydia and gonorrhea (by means of a specific polymerase-chain-reaction [PCR] assay performed on anal and throat swabs and urine samples). Treatment of incident sexually transmitted infections was provided according to the protocol recommendations. Postexposure prophylaxis was readily available at study sites in case of unprotected exposure to a possibly HIV-infected partner.

Primary End Point

The primary end point was the diagnosis of HIV-1 infection, which was defined as the first evidence of HIV antibodies or p24 antigen in serum with the use of a fourth-generation enzyme-linked immunosorbent assay (ELISA) for HIV-1 and HIV-2 combined or HIV-1 RNA in plasma on PCR assay. At most of the sites, investigators used the Architect HIV Ag/Ab Combo assay (Abbott) for ELISA and the RealTime HIV-1 assay (Abbott) or Cobas TaqMan HIV-1 Test, version 2.0 (Roche), for HIV RNA PCR. Genotypic testing for drug resistance was performed on the sample obtained at the time of diagnosis to detect major resistance mutations at positions 184, 65, and 70 of the reverse transcriptase gene.

Analysis of Adherence

Pill count was the first measure of adherence. Participants were asked to return their study-drug bottles at each visit, and a pill count of unused medication was performed. We also measured drug levels in plasma in the first participants who were enrolled. Plasma was tested for the presence of tenofovir and FTC with the use of a validated liquid chromatography-tandem mass spectrometry method with a limit of detection of 0.1 ng per milliliter for tenofovir and 0.4 ng per milliliter for FTC. This plasma assay was able to detect drugs up to 9 days after intake.

Adherence to the study regimen during the most recent sexual intercourse was also assessed by means of computer-assisted structured interviews that were completed before each visit. Three categories of adherence were defined: correct use of pre-exposure prophylaxis (at least one pill taken within 24 hours before sex and one pill taken within 24 hours after sex), no use of pre-exposure prophylaxis (no pills taken within 48 hours before and after sex), and suboptimal use of pre-exposure prophylaxis (i.e., any other use).

Safety

All participants who received at least one dose of TDF-FTC or placebo were included in the safety analyses. Adverse events were recorded at each visit, regardless of the perceived association with the medication. Toxicity was graded according to the scale of the severity of adverse events in adults used by the France Recherche Nord et Sud Sida-HIV et Hépatites (National Agency of Research on AIDS and Viral Hepatitis [ANRS]).

Study Oversight

The conduct of the trial at each study site was monitored by the Service Commun 10-Unité de Service 19 (a clinical trial center) of INSERM. Gilead Sciences donated the study medications and provided funding for the pharmacokinetics analysis but had no role in data collection, data analysis, or manuscript preparation. All the authors vouch for the completeness and accuracy of the data reported and adherence to the study protocol.

Statistical Analysis

We calculated that 64 HIV-1 seroconversion events would provide a power of 80% to detect a 50% relative reduction in the incidence of HIV-1 infection in the TDF-FTC group, as compared with the placebo group, at a two-sided alpha level of 0.05. The expected incidence of HIV-1 infection in the placebo group was 3 cases per 100 person-years. We determined that a sample size of 1900 participants would be required to achieve the target number of study end points, with 12 to 36 months of follow-up for each participant and a rate of loss to follow-up of 15 per 100 person-years.

We used a modified intention-to-treat approach for the primary analysis, in that we excluded data only from participants who were found to have HIV-1 infection before receiving the first dose of study medication or who were lost to follow-up or withdrew consent between randomization and enrollment and did not receive study medication. All participants who underwent randomization were included in an intention-to-treat analysis.

We used the Kaplan-Meier method to estimate the cumulative probability of HIV-1 infection per group and used the log-rank test to perform between-group comparisons. To assess sexual behavior over time in the two study groups, probit mixed models and binomial mixed models were used.

The study data were reviewed every 6 months by an independent data and safety monitoring board. On Oct. 23, 2014, just after the early discontinuation of another trial of preexposure prophylaxis involving men who have sex with men, called the Preexposure Option for Reducing HIV in the UK: An Open-Label Randomization to Immediate or Deferred Daily Truvada for HIV-Negative Gay Men (PROUD) in the United Kingdom, 19 the data and safety monitoring board asked for a first unblinded interim analysis of the data and subsequently recommended that the placebo group be discontinued and that all the study participants be offered on-demand preexposure prophylaxis. The present analysis includes data collected during the double-blind phase of the study up to Jan. 27, 2015. The study is now ongoing with an open-label design.

All analyses were conducted with the use of Stata/SE software, version 12.1 (StataCorp), and SAS software, version 9.2 (SAS Institute). All P values and confidence intervals are two-sided.

Results:

Study Participants

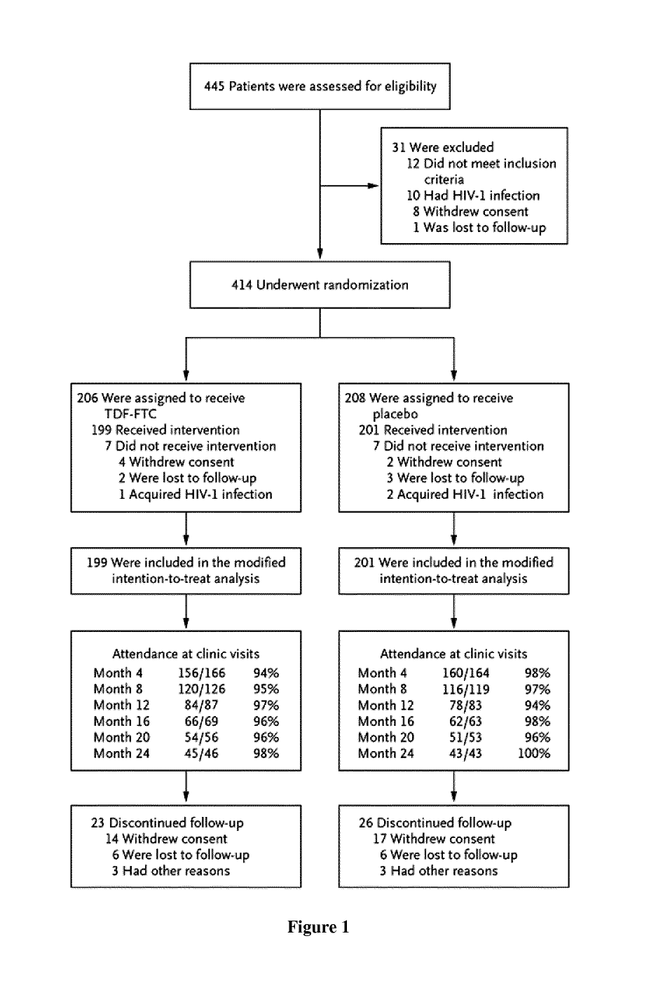

From Feb. 22, 2012, through Oct. 23, 2014, we screened 445 participants at seven study sites (six in France and one in Canada), which were opened sequentially during the study. Of the 414 participants who underwent randomization, 400 who subsequently tested negative for HIV infection were enrolled and followed during the study period (FIG. 1). Of the 400 participants, 56 (14%) (31 in the TDF-FTC group and 25 in the placebo group, P=0.37) received postexposure prophylaxis during the study period.

Retention was good during the study period, with premature study discontinuation by 49 participants (12%), for a total of 431.3 person-years of follow-up for the assessment of the incidence of HIV-1 infection after enrollment, with a median follow-up of 9.3 months (interquartile range, 4.9 to 20.6).

Adherence to Study Medication

Participants took a median number of 15 pills (interquartile range, 11 to 21) per month in the TDF-FTC group and 15 pills (interquartile range, 9 to 21) per month in the placebo group (P=0.57). Individual patterns of pill use showed large interpatient and intrapatient variability over time.

We also measured tenofovir and FTC levels in plasma for the first 113 participants who were enrolled. In the TDF-FTC group, the rates of detection were 86% for tenofovir and 82% for FTC, respectively, a finding that was consistent with receipt of each drug within the previous week. Tenofovir and FTC were also detected in 8 participants in the placebo group, 3 of whom were receiving postexposure prophylaxis.

Finally, we used computer-assisted structured interviews to analyze self-reports of the use of preexposure prophylaxis during the most recent sexual intercourse. Overall, 28% of participants did not take TDF-FTC or placebo, 29% took the assigned drug at a suboptimal dose, and 43% took the assigned drug correctly.

Sexual Behavior

Sexual practices did not change overall among the participants during the study period as compared with baseline. There were no significant between-group differences in the total number of episodes of sexual intercourse in the 4 weeks before visits (P=0.07), in the proportion of episodes of receptive anal intercourse without condoms (P=0.40), or in the proportion of episodes of anal sex without condoms during the most recent sexual intercourse (P=0.90). However, there was a slight but significant decrease in the number of sexual partners within the past 2 months in the placebo group as compared with the TDF-FTC group (7.5 and 8, respectively; P=0.001). The proportions of participants with a new sexually transmitted infection (of the throat, anus, and urinary tract combined) during follow-up were similar, with 41% in the TDF-FTC group and 33% in the placebo group (P=0.10). Most of the sexually transmitted infections (39%) were rectal infections. Overall, 81 participants (20%) acquired chlamydia infections during follow-up, 88 (22%) gonorrhea, 39 (10%) syphilis, and 5 (1%) hepatitis C virus. No participant acquired hepatitis B virus infection.

Effect of TDF-FTC on HIV-1 Acquisition

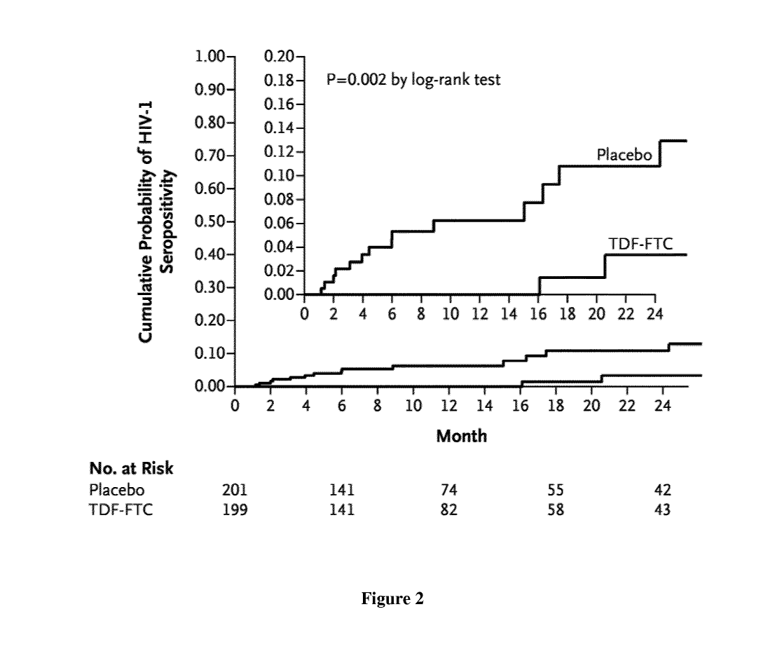

Overall, HIV-1 seroconversion was observed in 19 participants, of whom 3 acquired HIV-1 between randomization and enrollment. In the modified intention-to-treat analysis, 16 HIV-1 infections developed after enrollment: 2 in the TDF-FTC group (incidence of 0.91 per 100 person-years) and 14 in the placebo group (incidence of 6.60 per 100 person-years), indicating a relative reduction in the incidence of HIV-1 acquisition in the TDF-FTC group of 86% (95% confidence interval [CI], 40 to 98; P=0.002) (FIG. 2). In the intention-to-treat analysis, the relative reduction in the incidence of HIV-1 acquisition was 82% (95% CI, 36 to 97; P=0.002). The 2 participants in the TDF-FTC group in whom HIV-1 infection was diagnosed at scheduled visits returned 60 and 58 pills out of 60, respectively, at these visits and were therefore deemed to be nonadherent to preexposure prophylaxis. Study drugs were not detected in plasma samples obtained from these 2 participants at the time of HIV-1 diagnosis. None of the 16 participants who acquired HIV-1 infection after enrollment had resistance mutations to study medications.

Safety and Adverse Events

There were no significant between-group differences in the frequency of serious adverse events or grade 3 or 4 adverse events, and there were no deaths during the study. Only one participant in the TDF-FTC group discontinued the study drug because of a suspected drug-drug interaction with dabigatran when he presented with a relapse of deep venous thrombosis. Drug-related gastrointestinal adverse events (nausea, vomiting, diarrhea, abdominal pain, and other gastrointestinal disorders) were seen more commonly in the TDF-FTC group than in the placebo group (14% vs. 5%, P=0.002).

Elevations in serum creatinine levels were seen in 35 participants (18%) in the TDF-FTC group and 20 participants (10%) in the placebo group (P=0.03). All but one of these events were grade 1 and none led to study-drug discontinuation. Only 2 participants (1%), both of whom were in the TDF-FTC group, had a transient decrease in creatinine clearance to below 60 ml per minute.

Discussion:

In this study involving high-risk men who have sex with men, sexual activity-dependent preexposure prophylaxis with TDF-FTC was associated with a relative reduction of 86% in the risk of HIV-1 infection. This finding is among the highest risk reductions that have been reported to date, but the short follow-up for our study may have increased the likelihood of an exaggerated estimate of efficacy due in part to high initial adherence.19 In the iPrex trial involving young men who have sex with men, in which overall adherence to daily preexposure prophylaxis on the basis of drug testing was only 51%, the relative reduction in the risk of HIV-1 infection was 42% in the intention-to-treat analysis but increased to 92% in a case-control subgroup of participants with detectable levels of tenofovir in their blood.6 However, participants might not need continuous exposure to antiretroviral drugs to be protected from infection, especially when they are not exposed to HIV-1. In macaques, intermittent oral preexposure prophylaxis with TDF-FTC was shown to be at least as effective as daily prophylaxis.20,21 On the basis of data from studies in animals, we hypothesized that preexposure prophylaxis take at the time of sexual activity would provide adequate protection against HIV-1, while improving convenience and adherence to the drug regimen. Post hoc analyses from the open-label extension of the iPrex study have suggested that there were no incident HIV-1 infections among participants with an intracellular level of tenofovir diphosphate that was associated with continuous receipt of at least 4 tablets of TDF-FTC per week. This finding is consistent with the efficacy reported in our study, in which participants took a median of 15 pills per month. On the other hand, our results cannot be extrapolated to persons taking a lower number of pills per month. Assessing adherence to sexual activity-dependent preexposure prophylaxis is challenging and represents another limitation of our study. Measures of plasma drug levels revealed that a high proportion of participants in the TDF-FTC group were exposed to TDF-FTC. However, using selfadministered questionnaires to assess the use of preexposure prophylaxis at the time of the most recent sexual intercourse, we found that 28% of participants reported no use of such prophylaxis, suggesting that they were able to discern when to use preexposure prophylaxis on the basis of their own assessment of risk. It was reassuring that there was no obvious increase in behavior associated with heightened risk during follow-up in our study, a finding that was echoed in previous trials of preexposure prophylaxis. The use of TDF-FTC was associated with gastrointestinal symptoms and transientincreases in creatinine, both of which were consistent with previous reports.6 We were unable to assess the potential of long-term toxicity of TDF-FTC, and ultimately safety concerns will have to be balanced against potential benefits from HIV-1 prevention. In conclusion, our study showed a reduced incidence of HIV-1 infection with sexual activity—among high-risk men who have sex with men and who engage in unprotected anal sex. Given that participants took a median of 15 pills per month, the results of this study cannot be extrapolated to men who have sex with men who have less frequent sexual intercourse and thus would be taking TDF-FTC on a more intermittent regimen. While we wait for an effective vaccine against HIV, the use of such preexposure prophylaxis with TDF-FTC among high-risk men could contribute to a reduced incidence of HIV infection.

### REFERENCES

Throughout this application, various references describe the state of the art to which this invention pertains. The disclosures of these references are hereby incorporated by reference into the present disclosure.

UNAIDS. The gap report (http://www.unaids.org/en/resources/documents/2014/20140716_UNAIDS_gap_report).

Hammer S M, Sobieszczyk M E, Janes H, et al. Efficacy trial of a DNA/rAd5 HIV-1 preventive vaccine. N Engl J Med 2013; 369: 2083-92.

Auvert B, Taljaard D, Lagarde E, Sobngwi-Tambekou J, Sitta R, Puren A. Randomized, controlled intervention trial of male circumcision for reduction of HIV infection risk: the ANRS 1265 Trial. PLoS Med 2005; 2(11): e298.

Cohen M S, Chen Y Q, McCauley M, et al. Prevention of HIV-1 infection with early antiretroviral therapy. N Engl J Med 2011; 365: 493-505.

Connor E M, Sperling R S, Gelber R, et al. Reduction of maternal-infant transmission of human immunodeficiency virus type 1 with zidovudine treatment. N Engl J Med 1994; 331: 1173-80.

Grant R M, Lama J R, Anderson P L, et al. Preexposure chemoprophylaxis for HIV prevention in men who have sex with men. N Engl J Med 2010; 363: 2587-99.

Thigpen M C, Kebaabetswe P M, Paxton L A, et al. Antiretroviral preexposure prophylaxis for heterosexual HIV transmission in Botswana. N Engl J Med 2012; 367: 423-34.

Choopanya K, Martin M, Suntharasamai P, et al. Antiretroviral prophylaxis for HIV infection in injecting drug users in Bangkok, Thailand (the Bangkok Tenofovir Study): a randomised, double-blind, placebo-controlled phase 3 trial. Lancet 2013; 381: 2083-90.

Baeten J M, Donnell D, Ndase P, et al. Antiretroviral prophylaxis for HIV prevention in heterosexual men and women. N Engl J Med 2012; 367: 399-410.

Marrazzo J M, Ramjee G, Richardson B A, et al. Tenofovir-based preexposure prophylaxis for HIV infection among African women. N Engl J Med 2015; 372: 509-18.

Van Damme L, Corneli A, Ahmed K, et al. Preexposure prophylaxis for HIV infection among African women. N Engl J Med 2012; 367: 411-22.

Le Vu S, Le Strat Y, Barin F, et al. Population-based HIV-1 incidence in France, 2003-08: a modelling analysis. Lancet Infect Dis 2010; 10: 682-7.

Lavoie E, Alary M, Remis R S, et al. Determinants of HIV seroconversion among men who have sex with men living in a low HIV incidence population in the era of highly active antiretroviral therapies. Sex Transm Dis 2008; 35: 25-9.

Beyrer C, Sullivan P, Sanchez J, et al. The increase in global HIV epidemics in MSM. AIDS 2013; 27: 2665-78.

Kamb M L, Fishbein M, Douglas J M Jr, et al. Efficacy of risk-reduction counseling to prevent human immunodeficiency virus and sexually transmitted diseases: a randomized controlled trial. JAMA 1998; 280: 1161-7.

Descamps D, Delaugerre C, Masquelier B, et al. Repeated HIV-1 resistance genotyping external quality assessments improve virology laboratory performance. J Med Virol 2006; 78: 153-60.

J, Loze B, Morel S, et al. Tenofovir and emtricitabine pharmacokinetics in plasma and saliva following a single dose of TDF 600 mg/FTC 400 mg: implications of on demand PrEP (ANRS Ipergay). Presented at the 20th International AIDS Conference, Melbourne, VIC, Australia, July 20-25, 2014. abstract.

ANRS scale to grade the severity of adverse events in adults. Version 1.0 4. November 2008 (http://www.anrs.fr/Rubriques-transversales/Outils-pour-la-recherche).

McCormack S, Dunn D T, Desai M, et al. Preexposure prophylaxis to prevent the acquisition of HIV-1 infection (PROUD): effectiveness results from the pilot phase of a pragmatic open-label randomised trial. Lancet 2015 September 9 (Epub ahead of print).

Garcia-Lerma J G, Otten R A, Qari S H, et al. Prevention of rectal SHIV transmission in macaques by daily or intermittent prophylaxis with emtricitabine and tenofovir. PLoS Med 2008; 5(2): e28.

Garcia-Lerma J G, Cong M E, Mitchell J, et al. Intermittent prophylaxis with oral Truvada protects macaques from rectal SHIV infection. Sci Transl Med 2010; 2: 14ra4.

Grant R M, Anderson P L, McMahan V, et al. Uptake of pre-exposure prophylaxis, sexual practices, and HIV incidence in men and transgender women who have sex with men: a cohort study. Lancet Infect Dis 2014; 14: 820-9.

Youle M, Wainberg M A. Could chemoprophylaxis be used as an HIV prevention strategy while we wait for an effective vaccine? AIDS 2003; 17: 937-8.

1. A method for on demand pre-exposure prophylactic treatment of a subject at risk for HIV infection comprising administering orally to the subject a combination of a therapeutically effective amount of emtricitabine and tenofovir prior to and after exposure to a potential source of HIV.

2. The method of claim 1 wherein the potential source of HIV is sexual intercourse.

3. The method of claim 1 wherein the subject is in an ongoing relationship with an HIV-positive partner.

4. The method of claim 1 wherein the subject
1) is not in a mutually monogamous relationship with a partner who recently tested HIV-negative,
2) is a gay or bisexual man who has had anal sex without a condom or been diagnosed with an STD in the past 6 months; or
3) is a heterosexual man or woman who does not regularly use condoms during sex with partners of unknown HIV status and who are at risk of HIV infection.

5. The method of claim 1 wherein the subject is a man who has sex with another man.

6. The method of claim 1 wherein the tenofovir and the emtricitabine are combined in one pill.

7. The method of claim 6 wherein the pill comprises 300 milligrams of tenofovir disoproxil fumarate and 200 milligrams of emtricitabine.

8. The method of claim 1 wherein a first dose of the combination is administered 24 h before the exposure, a second dose is administered 24 h after the first dose and a third dose is administered after 48 h after the first dose.

9. The method of claim 8 wherein the first dose is two pills and the second dose and the third dose are each one pill.
